(19) United States
(12) Patent Application Publication
(10) Pub. No.: US 2025/0114632 A1
(43) Pub. Date: Apr. 10, 2025

(54) METHODS, SYSTEMS AND DEVICES FOR USING GREEN LIGHT FOR NEURODEGENERATIVE DISEASES AND OTHER CONDITIONS WITH INFLAMMATORY REPONSES

(71) Applicant: Arizona Board of Regents on Behalf of the University of Arizona, Tucson, AZ (US)

(72) Inventors: Mohab Ibrahim, Tucson, AZ (US); Laurent Martin, Tucson, AZ (US)

(21) Appl. No.: 18/730,490

(22) PCT Filed: Jan. 23, 2023
(86) PCT No.: PCT/US2023/061096

§ 371 Date: Jul. 19, 2024

Related U.S. Application Data
(60) Provisional application No. 63/301,896, filed on Jan. 21, 2022.

(51) Int. Cl. A61N 5/06

(52) U.S. Cl. CPC A61N 5/0618; A61N 5/0613; A61N 2005/0626; A61N 2005/0648; A61N 2005/0651; A61N 2005/0663; A61N 2005/0667

(57) ABSTRACT

Methods, systems and devices for administering green light to subjects are described. The described techniques can be used for managing cognitive deficits or memory impairments, reducing the symptoms associated with posttraumatic stress disorder (PTSD), anxiety and neurodegenerative diseases and other conditions. One example method for managing or minimizing cognitive deficits or memory impairments includes administering, for a first time period and according to a pre-arranged schedule of administrations, light with one or more wavelengths in the range between approximately 515 nm and approximately 535 nm to a retina of a subject. The method further includes administrating the light, for a second time and subsequent periods according to the pre-arranged schedule of administrations to the retina of the subject. The first, the second and the subsequent time periods occur on separate days, and the pre-arranged schedule of administrations includes administrations that span over at least one week.

### CROSS-REFERENCE TO RELATED APPLICATION(S)

This application claims priority to the provisional application with Ser. No. 63/301,896 titled “METHODS, SYSTEMS AND DEVICES FOR USING GREEN LIGHT FOR NEURODEGENERATIVE DISEASES AND OTHER CONDITIONS WITH INFLAMMATORY REPONSES,” filed Jan. 21, 2022. The entire contents of the above noted provisional application are incorporated by reference as part of the disclosure of this document.

### STATEMENT REGARDING FEDERALLY SPONSORED RESEARCH

This invention was made with government support under Grant No. AT009716 awarded by the National Institutes of Health. The government has certain rights in the invention.

### TECHNICAL FIELD

The technology in this patent document relates to administering light therapy, and specifically green light therapy, for managing symptoms associated with various conditions.

### SUMMARY

Methods, systems and devices are described that administer green light to subjects for managing or minimizing cognitive deficits or memory impairments, as well as reducing the symptoms associated with posttraumatic stress disorder (PTSD), anxiety and neurodegenerative diseases and other conditions, as described herein.

One aspect of the disclosed embodiments relates to a method for managing or minimizing cognitive deficits or memory impairments that includes administering, for a first time period and according to a pre-arranged schedule of administrations, light with one or more wavelengths in the range between approximately 515 nm and approximately 535 nm to a retina of a subject. The method further includes administrating, for a second time and subsequent periods according to the pre-arranged schedule of administrations, light with the one or more wavelengths in the range between approximately 515 nm and approximately 535 nm to the retina of the subject. The first, the second and the subsequent time periods occur on separate days, and the pre-arranged schedule of administrations includes administrations that span over at least one week.

### BRIEF DESCRIPTION OF THE DRAWINGS

FIG. 1 illustrates, in panel A, representative cytokine array and, in panel B, quantification of different cytokine levels from male rats exposed to white or green light based on experiments conducted in accordance with example embodiments.

FIG. 2 illustrates swimming speed and visual perception of rats after exposure to green and white light based on experiments conducted in accordance with example embodiments.

FIG. 3 illustrates example effects of exposure to green light on transgenic mice, including representative paths in a session of hidden platform training and learning curves during spatial training in the Morris water maze based on experiments conducted in accordance with example embodiments.

FIG. 4 illustrates the impact of green light exposure on memory dysfunctions based on experiments conducted in accordance with example embodiments.

FIG. 5 illustrates an example green light exposure procedure the impact of green light exposure on anxiety-like behavior based on experiments conducted in accordance with example embodiments.

FIG. 6 illustrates an example green light exposure procedure the impact of green light exposure on posttraumatic stress disorder symptoms based on experiments conducted in accordance with example embodiments.

FIG. 7 illustrates a set of operations that can be carried out for managing or minimizing cognitive deficits or memory impairments in accordance with an example embodiment.

FIG. 8 illustrates a set of operations that can be carried out for modulating microglia activation in a subject in accordance with an example embodiment.

FIG. 9 illustrates a set of operations that can be carried out for reducing symptoms associated with posttraumatic stress disorder (PTSD).

FIG. 10 illustrates an eyewear system for administering green light exposure in accordance with an example embodiment.

### DETAILED DESCRIPTION

In this document the term green light emitting diode (GLED) is used by the way of example, and not by limitation, to convey that light emitting diodes can be used as light sources to emit light in the suitable range of wavelengths. Further, the terms GLED or GLED therapy in this document are sometimes used to refer to green light therapy.

We have shown that green light therapy has great benefits in patients suffering from chronic pain in not only reducing or alleviating the pain, but in also managing or reducing drug abuse disorders because green light reduces the amount of drug required for pain treatment. Similar benefits can also be reaped in treating alcohol abuse which involves considerable neuroinflammation. Thus, through acting on neuroinflammation, GLED can help patients to recover from alcohol abuse. Notably, the underlying mechanisms suggest a high potential of GLED for the treatment of other diseases involving inflammation and cognitive impairments. Our previous projects focusing on the antinociceptive effect of green light therapy allowed us to identify that exposure to green light can modulate the inflammatory system by decreasing microglia activation. Microglia are macrophage-like immune cells in the central nervous system that maintain brain homeostasis. In response to immunological stimulus, microglial cells adopt an amoeboid morphology and release interleukins (IL) like IL-1β and IL-6 and tumor necrosis factor-α (TNFα). Microglia is typically considered as exhibiting dual phenotypes when activated. In particular, M1 is considered the classically activated state, M2 is the alternately activated state. The M1 phenotype is involved in neuroinflammation and is neurotoxic, whereas the M2 phenotype is neuroprotective. However, the microglial activation profile has been recently characterized as having more of a gray scale, depending on the conditions; that is, a broad array of activation profiles and phenotypes has been recently described, especially in relation to the neurodegenerative diseases.

Inflammatory processes are highly involved in the evolution and development of diseases such as Alzheimer's, Parkinson's, or multiple sclerosis. By controlling these inflammatory processes, GLED presents strong benefits to treat patients affected by these neurodegenerative diseases. Furthermore, our preliminary data revealed that GLED increases an inhibitor of a protein that is involved in toxic amyloid-β oligomer production, a hallmark of Alzheimer's disease. The disclosed embodiments present the benefits of GLED in reducing the symptoms of neurodegenerative diseases and improving cognitive impairments. Additionally, since green light has been shown to modulate microglia from a hyper-activated state (harmful) to a restive state (helpful), the disclosed technology can be applied to treat or alleviate other diseases or conditions with characteristic inflammatory responses carried out by microglial cells, including neurodegenerative diseases such as Alzheimer's, Parkinson's, multiple sclerosis, as well as other conditions, such as severe acute respiratory syndrome coronavirus-2 (SARS-CoV-2), which according to recent studies infects microglia and induces its subsequent activation and transformation into a pro-inflammatory M1 phenotype. Additionally, green light exposure is beneficial for treating depression and anxiety, including postpartum depression, which studies have shown to be associated with heightened activation of the microglia.

Neurodegenerative diseases are the most common cause of dementia and one of the leading causes of morbidity and mortality in the aging population. They present cognitive dysfunction, anxiety, and language deficits, leading to a significant socio-economic burden with our ever-aging population. Currently, the management of Alzheimer's disease relies on symptomatic control. Cholinesterase inhibitors and NMDA receptor antagonists have been used. However, current therapies for neurodegenerative diseases are not sufficient. Therefore, there is a need for new methods to prevent, delay, or manage their symptoms.

The pathological hallmarks of Alzheimer's disease (AD) are extracellular insoluble deposits of amyloid-β protein (Aβ), which form senile plaques, together with intracellular neurofibrillary tangles of hyperphosphorylated tau proteins. Along these lines, compelling evidence has demonstrated that soluble oligomers of Aβ (Aβo) are more toxic to neurons and synapses than insoluble Aβ forms. Aβ oligomers are the products of amyloid-β precursor protein (APP) cleavage. Their toxicity is dependent on oligomerization levels. Multiple proteins are involved in the cleavage of APP, such as β- and γ-secretases, but also metalloproteases (MMPs). Notably, MT5-MMP plays a crucial role in generating highly toxic Aβo.

However, the failure of clinical trials that attempted to modulate Aβo production suggests that other factors may influence the development and maintenance of AD. It has been speculated that chronic inflammation causes dysregulation of the Aβ clearance mechanisms. Aβo trigger an immune response with persistent activation of microglia, neuroimmune cells, that causes neuronal degeneration and contributes to disease progression and severity. Importantly, our studies demonstrate that exposure to green light emitting diodes (GLED) decreases microglial activation in the spinal cord. Therefore, GLED may also decrease microglial activation in the brain, which in turn may decrease chronic inflammation and neurodegeneration processes.

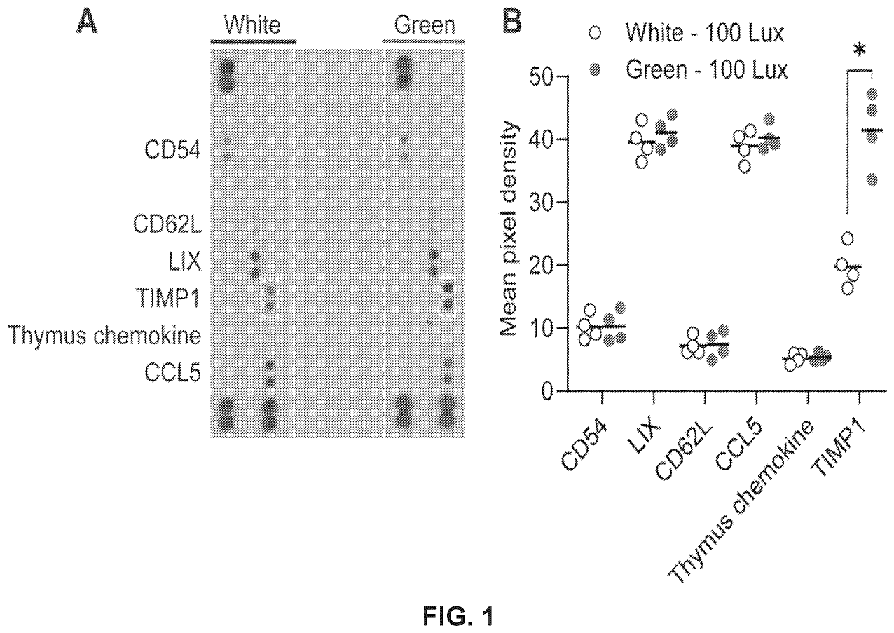

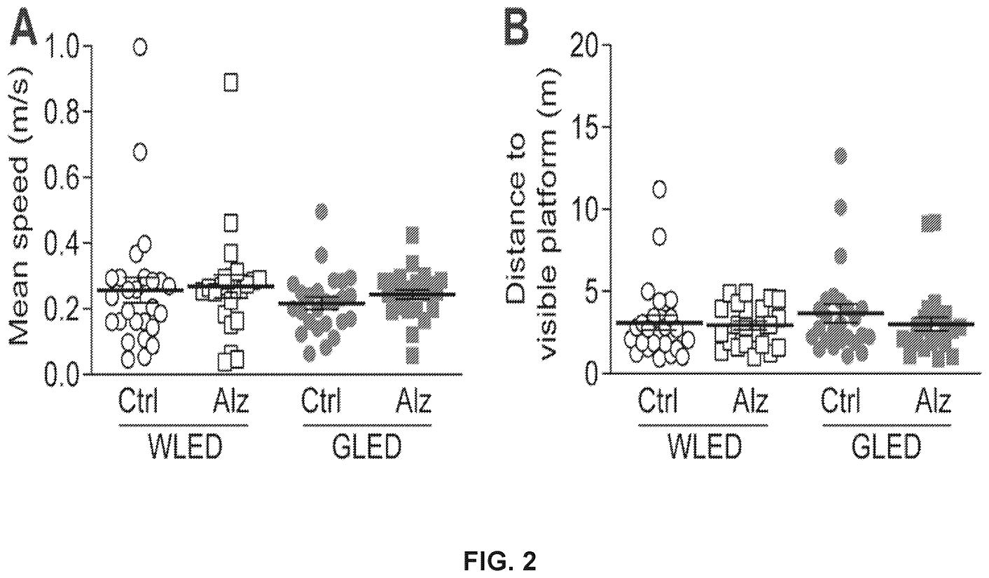

Our current work with green light emitting diodes has shown that GLED exposure increases TIMP-1 levels, an inhibitor of MMPs, in the cerebrospinal fluid (CSF), as illustrated by the example plots in FIG. 1. This figure shows representative cytokine array (A) and quantification (B) of different cytokine levels from male rats (n=4 independent experiments) exposed to white or green light. Notably, TIMP-1 also inhibits ADAM10 activity, another secretase involved in APP processing. As we observed an increased in TIMP-1 expression, we hypothesized that GLED exposure may also reduce toxic oligomer synthesis and thus cognitive impairments. Consequently, we analyzed the potential benefits of GLED using a Morris water maze test, evaluating cognitive impairments. Importantly, GLED did not affect motor abilities of visual perception, as illustrated in FIG. 2. In FIG. 2, example swimming speed and visual perception after exposure to green light are presented for N=23-26, 3 independent experiments, where results are presented as mean±SEM. Panel (A) illustrates average swimming speed in a Morris water maze after exposure (1-3 weeks) to white (WLED) or green light (GLED). In all our groups, the light exposure did not affect swimming abilities. The J20 mice (Alz in FIG. 2) did not present any motor impairments compared to the control group (Ctrl). Panel (B) illustrates mean distance travelled by the mice to reach a visible platform in a Morris water maze. After light exposure protocols, none of our groups presented any impairments in their ability to locate a visible platform.

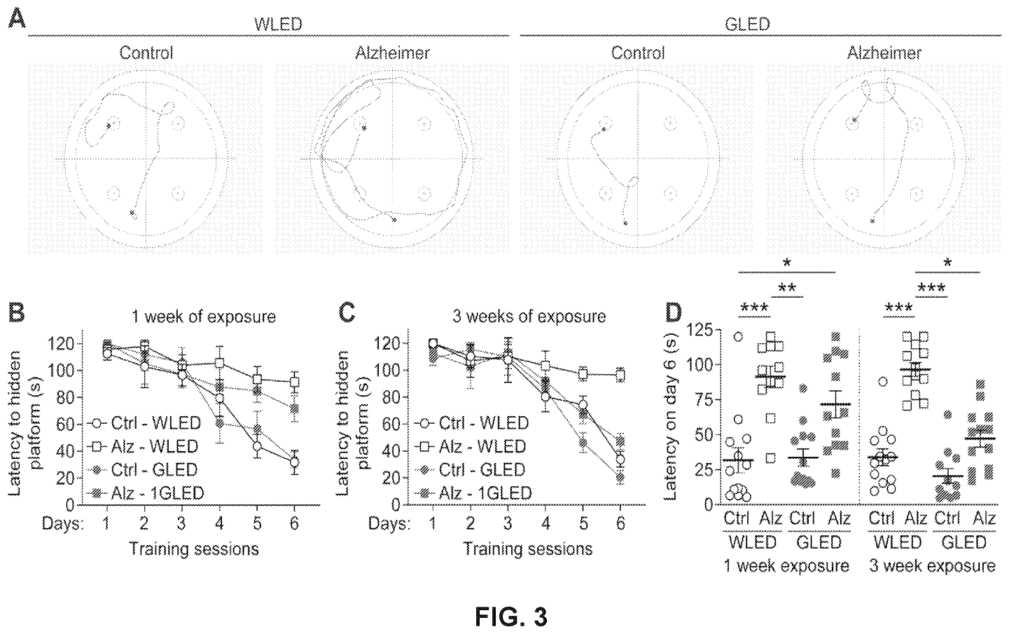

To evaluate the impact of green light exposure on memory deficits, we used J20 transgenic mice. This strain presents spatial memory impairments and senile plaques, hallmark of Alzheimer's disease. We analyzed learning deficits by training the mice over 6 days in the maze. J20 mice showed little ability to learn the task well, even when exposed to GLED for a week. Nevertheless, when J20 mice were exposed to GLED for 3 weeks, their latency to reach the hidden platform decreased compared to J20 mice exposed to WLED, as illustrated in FIG. 3. In this figure, example effects of GLED on the phenotype of J20 transgenic mice are presented (mean±SEM). Panel (A) illustrates representative paths from the last session of hidden platform training in a Morris water maze, where WLED represents exposure to white light and GLED represents exposure to green light. Panel (B) illustrates learning curves during spatial training in the Morris water maze. The latency for each mouse to reach the hidden platform was recorded. The curves illustrate the influence of GLED exposure (1 week) on spatial reference memory in the J20 model of cognitive deficits. Five-month-old WT and J20 mice were trained in the Morris water maze for 6 days. During acquisition of the task, escape latency was recorded. Two-way ANOVA did not reveal any significant effect of GLED after the mice were exposed for 1 week. J20 mice showed little ability to learn the task well. Panel (C) illustrates influence of GLED exposure (3 weeks) on spatial reference memory in the J20 model of cognitive deficits. Two-way ANOVA revealed a significant effect of GLED after the mice were exposure for 3 weeks. J20 mice exposed to GLED performed better than the J20 mice exposed to WLED, demonstrating improvements of spatial reference memory following GLED exposure. Panel (D) is an example histogram presenting the latency for each mouse to reach the hidden platform on day 6 of training. One week exposure to GLED did not result in significant improvement of cognitive functions, but mice exposed to GLED for 3 weeks demonstrated significant improvement of spatial reference memory (Kruskal Wallis test, n=11-13, obtained from 3 independent experiments).

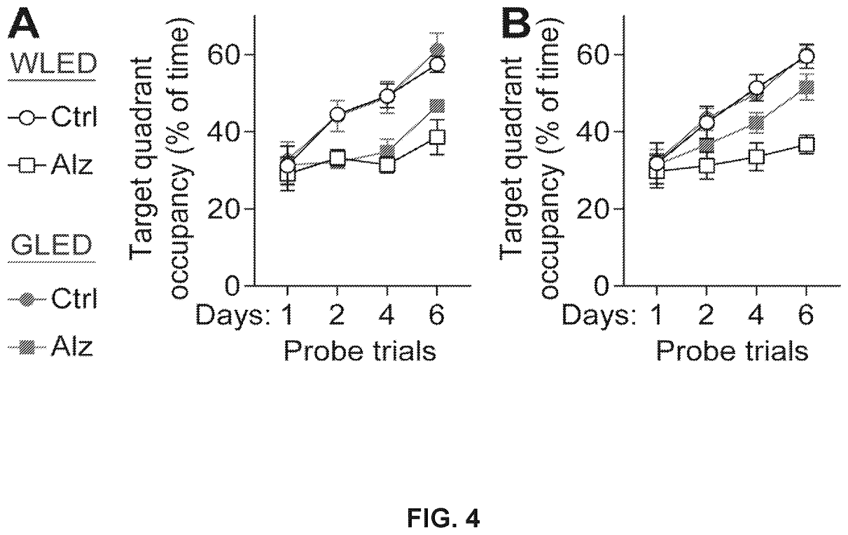

The GLED mice also spent more time in the target quadrant during probe trials, as illustrated in FIG. 4. In FIG. 4, GLED impact on memory dysfunctions is illustrated (two-way ANOVA, n=11-13, obtained from 3 independent experiments). Wild-type (Ctrl) and J20 mice were exposed to white (WLED) or green (GLED) light for 1 week (Panel (A)) or 3 weeks (Panel (B)). The mice were trained in a Morris water maze for 6 days. On days 1, 2, 4 and 6, the platform was removed to perform a probe trial, evaluating the time spent in the target quadrant, where the platform was previously located. Although J20 mice exposed to GLED for 1 week did not present any improvement of memory deficits compared to WLED-exposed mice, 3 weeks of GLED resulted in significant improvement of reference memory. These results demonstrate GLED can be used as a prophylactic measure to reverse or slow down the symptoms of neurodegenerative diseases such as Alzheimer's, Parkinson's, or multiple sclerosis.

The following experimental methods were used.

Cerebrospinal fluid (CSF) collection: Animals were anesthetized with 5% halothane and positioned in a stereotaxic frame. The head was flexed downward at approximately 45 degrees, a depressible surface with the appearance of a rhomb between occipital protuberances and the spine of the atlas becomes visible. A 23 G needle was punctured into the cisterna magna for CSF collection without making any incision at this region. The non-contaminated sample was drawn into a syringe by simple aspiration.

Morris water maze: The maze consisted of a pool (150-cm diameter) filled with water (21±1° C.) made opaque with nontoxic white tempera paint powder; the pool was located in a room surrounded by distinct extra-maze cues. Before hidden platform training, mice were given pre-training trials in which they had to swim in a rectangular channel (30 cm×60 cm). The day after pre-training, mice were trained in the circular water maze. For hidden platform training, the platform (10×10 cm) was submerged 1.5 cm below the surface. Mice received two training sessions with a 30-minute intertrial interval. The maximum time allowed per trial in this task was 120 sec. For probe trials, the platform was removed, and mice were allowed to swim for 60 sec before they were removed.

Some aspects of the disclosed embodiments relate to methods, devices and systems for managing or minimizing cognitive deficits and memory impairments. In particular, provided herein are systems and methods for managing and minimizing cognitive deficits and memory impairments with light therapy.

The disclosed GLED devices can be implemented using light sources that emit light in the green range of wavelengths (e.g., 525-535 nm within the larger range of 495-570 nm). In particular one or more light emitting devices (LEDs) may be used. Alternately, a light source with a broader emission bandwidth (e.g., a while light source) can be used with appropriate bandpass filters to selectively transmit the green range of wavelengths. GLED therapy can be implemented using light sources that are configured to produce ambient light. For example, the light sources can produce illumination for an enclosure (e.g., a room, a booth, etc.) that allows a patient to perceive the green light for particular durations over a period of time (e.g., 1 to 3 weeks). In some implementations, the light sources can be implemented as light bulbs. Additionally, or alternatively, the green light sources can be implemented as part of an eyewear device, including but not limited to, glasses, contact lenses, or goggles (e.g., ski goggles, virtual reality, augmented reality goggles, etc.). In some implementations, additional optical components, such as lenses, mirrors, prisms, and/or optical waveguides, may be used as part of the light source or as part of the eyewear to facilitate the delivery of the green light to the patient's eyes. The exposure rate, exposure duration and/or the intensity of the exposure can be controlled by an electronic device that is communicates with the light source(s) via a wired or wireless communication link.

Another aspect of the disclosed embodiments relates to mitigating the effect of posttraumatic stress disorder (PTSD) based on exposure to GLED. PTSD is a psychiatric disorder that occurs when a person faces a trauma or a set of circumstances that lead to functional and cognitive impairments. PTSD is one of the serious health concerns associated with comorbidity and increased mortality with suicidal ideations and attempts. A person can experience this as a harmful emotional or life-threatening experience that can affect the well-being of the mind, body and society. Examples include natural disasters, major accidents, sexual abuse, terrorist acts, and combat.

Most research has focused on understanding and characterizing the underlying mechanisms of PTSD to develop new and effective treatments. For this purpose, multiple animal models have been developed, such as the single-prolonged stress model (SPS). SPS, which is used in the conducting the procedures disclosed herein, is a multimodal traumatic protocol based on a sequence of three stressors during a single session. This protocol reproduces psychological, physiological and pharmacological stress through restrainment, forced swim and ether inhalation, respectively. Seven days following induction, this model reproduces multiple features observed in PTSD, such as enhanced negative feedback of the Hypothalamic-Pituitary-Adrenal axis, cognitive impairments, strong anxiety, reduced social behavior and depression.

Photobiomodulation has been effective in treating multiple medical conditions, including depression and mood in adolescents taking antidepressants. Moreover, exposing patients to bright light decreased symptoms of seasonal affective disorder. Light therapy has also been implemented to increase daytime alertness by circadian stimulation, to improve sleep quality and to mitigate depression in Alzheimer's disease. Over the past decade, multiple studies have shown the benefits of green light in treating pain, and emerging results demonstrate a strong potential in treating anxiety. In the procedures described herein, we analyzed the effects of green light exposure to improve PTSD symptoms such as anxiety and panic.

Using the SPS model of PTSD and multiple exposure protocols, we demonstrated that green light can reverse PTSD-induced anxiety and even block its establishment. Our results also confirmed that exposure to green light reduces anxiety in naïve animals. These findings can be further extended to show that green light exposure also reduces generalized anxiety, panic disorders, and opioid use disorder.

Example Induction of PTSD: Following habituation to the experimenter, Sprague Dawley rats were exposed to three different stressors over a single session. First, animals were restrained in acrylic tubes for two hours. Then, the rats were forced to swim in a 70-cm bucket filled with water for 20 minutes. Finally, rats were placed in a box filled with ether until loss of consciousness.

Evaluation of anxiety methodology: An elevated plus maze was used to quantify anxiety behavior. Animals were placed in the center of the maze at the beginning of the testing session. Then, animals' tracks were recorded over 5 minutes, using a camera and AnyMaze Software. To reflect the anxiety index, a ratio between the time spent in the closed arm and the open arm was calculated for each animal.

Example light exposure: Animals were exposed to white or green LED (4-100 LUX) for 8 hours a day over a week. All visible spectrum LED flex strips were purchased from ledsupply.com (VT, USA). The specifications of the LEDs were: (i) #LS-AC50-GR-006, 525-nanometer wavelength (i.e., green), 8 Watts, 120 Volts, 120-degree beam angle; and (ii) #LS-AC50-WW-006, white, 9.6 Watts, 120 Volts, 120-degree beam angle. For exposure, LED strips were affixed on the top of racks where animals were housed, allowing global diffusion of light. Animals were exposed to the LED in these cages with full access to food and water in a dark room devoid of any other source of light.

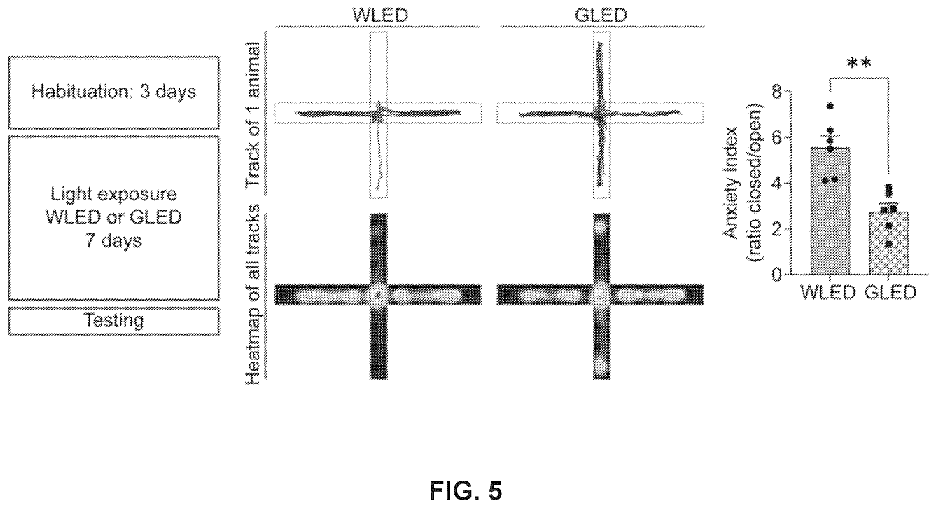

The results indicate that GLED decreases anxiety-like behavior in naïve animals, as illustrated in FIG. 5. As shown by the sequence of events on the left side, following 3-day habituation to the experimenter, Sprague-Dawley rats were exposed to white (control, WLED) or green (treatment, GLED) light-emitting diodes for 8 hours a day for seven days. At the end of exposure, anxiety-like behavior was evaluated using an elevated plus maze test by playing the rate at the center of the maze and recording their over 5 minutes, as shown in the middle panel of FIG. 5. The results showed that following exposure, GLED rats spent more time in the open arm compared to WLED rats, demonstrating reduced anxiety-like behavior (Mann Whitney non parametric test, n=6 per condition, **p<0.01), as demonstrated by the computed anxiety index in the right panel of FIG. 5.

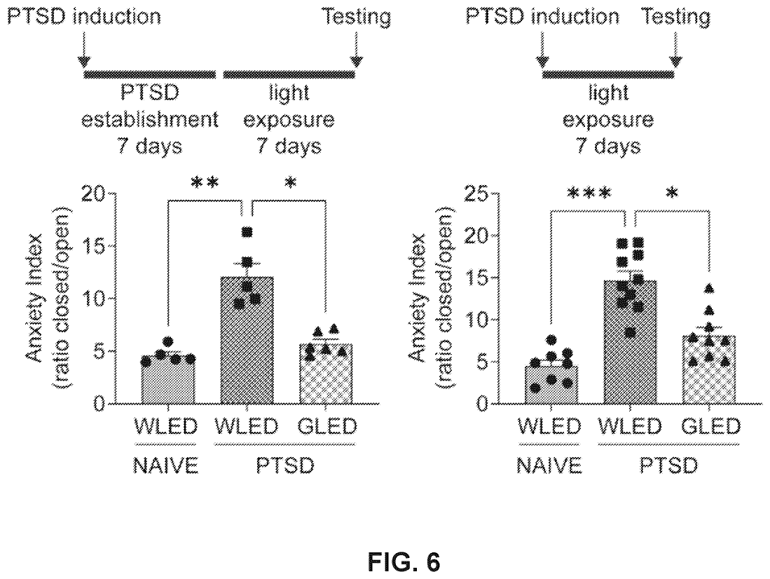

Our experiments further illustrate that green light exposure decreases PTSD symptoms in a rat model of PTSD, as illustrated in FIG. 6. In the procedure, following induction of PTSD through a single-prolonged stress protocol, Sprague-Dawley rats were exposed to white (control, WLED) or green (treatment, GLED) light-emitting diodes for 8 hours a day, starting 7 days after PTSD induction (left panel) or right after PTSD induction (right panel). At the end of exposure, anxiety-like behavior was evaluated using an elevated plus maze test. Following exposure, GLED rats spent more time in the open arm compared to WLED rats, demonstrating reduced anxiety-like behavior in a rat model of PTSD (Kruskal Wallis non parametric test, n=5-10 per condition, *p<0.05, **p<0.01, ***p<0.001).

The disclosed GLED administration can mitigate or reduce the symptoms of PTSD, anxiety, opioid use disorder, and panic disorder.

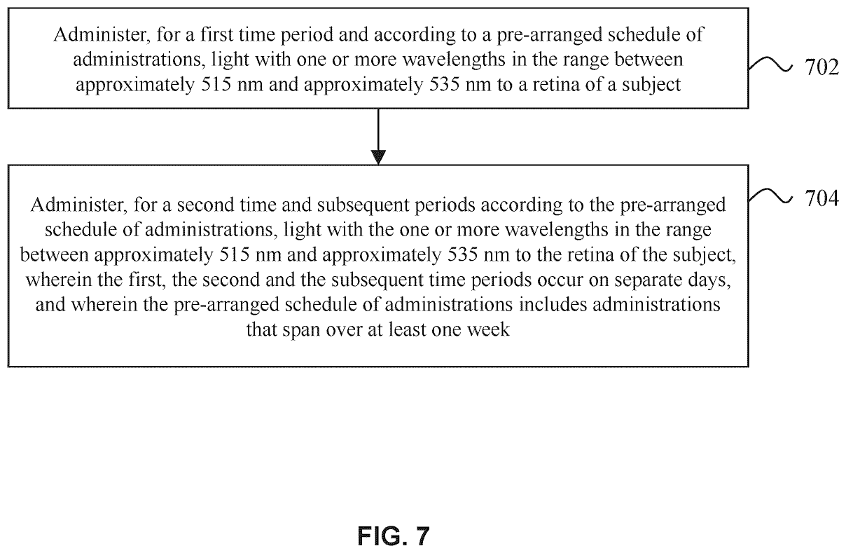

FIG. 7 illustrates a set of operations that can be carried out for managing or minimizing cognitive deficits or memory impairments in accordance with an example embodiment. The operation at 702 includes administering, for a first time period and according to a pre-arranged schedule of administrations, light with one or more wavelengths in the range between approximately 515 nm and approximately 535 nm to a retina of a subject. The operations at 704 includes administrating, for a second time and subsequent periods according to the pre-arranged schedule of administrations, light with the one or more wavelengths in the range between approximately 515 nm and approximately 535 nm to the retina of the subject. The first, the second and the subsequent time periods occur on separate days, and the pre-arranged schedule of administrations includes administrations that span over at least one week.

In one example embodiment, the first, the second and the subsequent time periods each occur on consecutive days. In another example embodiment, the first, the second and the subsequent time periods occur on consecutive days for more than one week. In still another example embodiment, the first, the second and the subsequent time periods occur on consecutive days that span three weeks.

According to one example embodiment, the administering comprises administering light of 0.001 lux to 1000 lux. In another example embodiment, the administering comprises administering light of approximately 100 lux. In yet another example embodiment, said administering includes contacting an eye of the subject with a material that allows light between approximately 515 nm and approximately 535 nm to enter the retina of the subject. In still another example embodiment, the material is a material that is configured to filter out light of a wavelength that is not between approximately 515 nm and approximately 535 nm.

In another example embodiment, the subject is affected by a neurogenerative disease, and administering the light for the first, the second and the subsequent time periods that span over at least one week is operable to mitigate cognitive deficits or memory impairments associated with the neurogenerative disease via modulating activation of microglia. According to another example embodiment, the neurogenerative disease is one or more of: Alzheimer's disease, Parkin's disease, or multiple sclerosis.

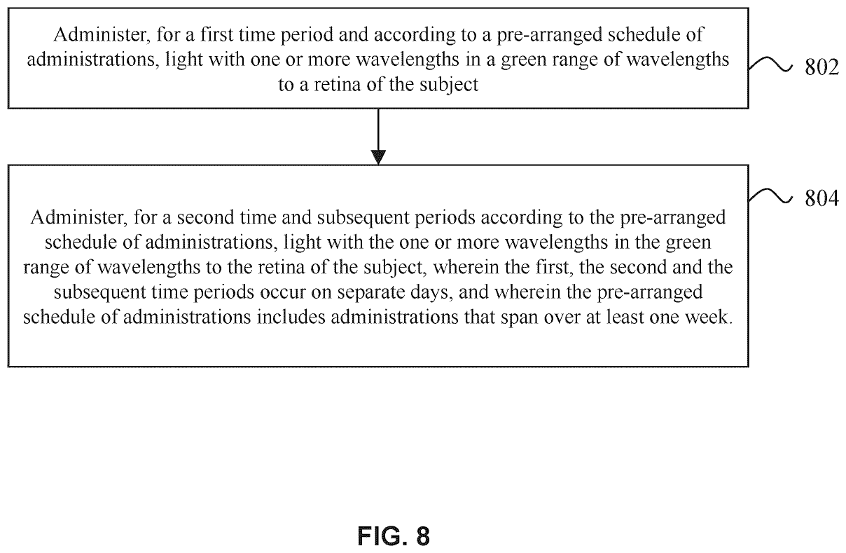

FIG. 8 illustrates a set of operations that can be carried out for modulating microglia activation in a subject in accordance with an example embodiment. The operations at 802 include administering, for a first time period and according to a pre-arranged schedule of administrations, light with one or more wavelengths in a green range of wavelengths to a retina of the subject. The operations at 804 include administrating, for a second time and subsequent periods according to the pre-arranged schedule of administrations, light with the one or more wavelengths in the green range of wavelengths to the retina of the subject. The first, the second and the subsequent time periods occur on separate days, and the pre-arranged schedule of administrations includes administrations that span over at least one week.

In one example embodiment of the method described in FIG. 8, modulating the microglia activation is operable to mitigate an inflammatory response associated with severe acute respiratory syndrome coronavirus-2 (SARS-CoV-2). In another example embodiment, modulating the microglia activation is operable to mitigate symptoms associated with one or more of: depression, anxiety, or postpartum depression.

One aspect of the disclosed embodiments relates to a device for microglia activation that includes one or more light sources having emission spectra within a range from approximately 515 nm to approximately 535 nm associated with green light. The device further includes a controller configured to control at least one of: intensity of light emission or duration of light emission of the one or more light sources. The device is configured to allow administration of the green light to a person wearing the device.

In one example embodiment, the device includes one of: goggles, ski goggles, virtual reality goggles, augmented reality goggles, a pair of eyeglasses, or a contact lens.

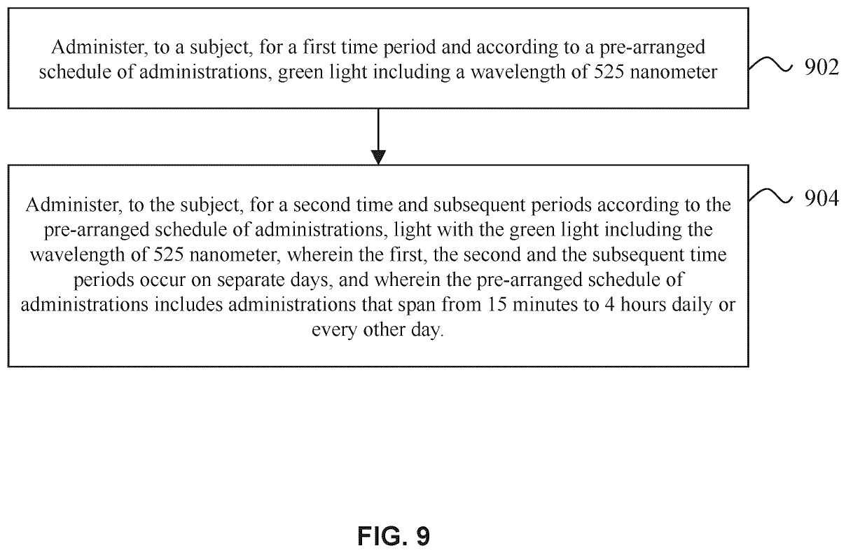

FIG. 9 illustrates a set of operations that can be carried out for reducing symptoms associated with posttraumatic stress disorder (PTSD). The operations at 902 includes administering to a subject, for a first time period and according to a pre-arranged schedule of administrations, green light including a wavelength of 525 nanometer. The operations at 904 includes administrating to the subject, for a second time and subsequent periods according to the pre-arranged schedule of administrations, the green light including the wavelength of 525 nanometer. The first, the second and the subsequent time periods occur on separate days, and the pre-arranged schedule of administrations includes administrations that span from 15 minutes to 4 hours daily or every other day.

In one example embodiment, the green light includes wavelengths in a range 500 to 550 nanometers. In another example embodiment, the administering comprises administering the green light of 0.001 lux to 500 lux intensity. In still another example embodiment, reducing the symptoms associated with posttraumatic stress disorder (PTSD) includes reducing anxiety associated with PTSD. In yet another example embodiment, the administering for the first time includes administering subsequent to onset of the PTSD symptoms. In another example embodiment, the administering for the first time, the second time or subsequent periods spans a period during which a stressful event that triggers the PTSD symptoms is occurring.

According to some embodiments, the above noted method includes delivering the green light through light-emitting glasses or goggles. In another example embodiments, the method includes delivering the green light through one or more lenses that only allow wavelengths in the green light spectra to pass therethrough. For example, the one or more lenses are one or more contact lenses. In yet another example embodiment, the method includes delivering the green light through a light source that illuminates an environment around the subject.

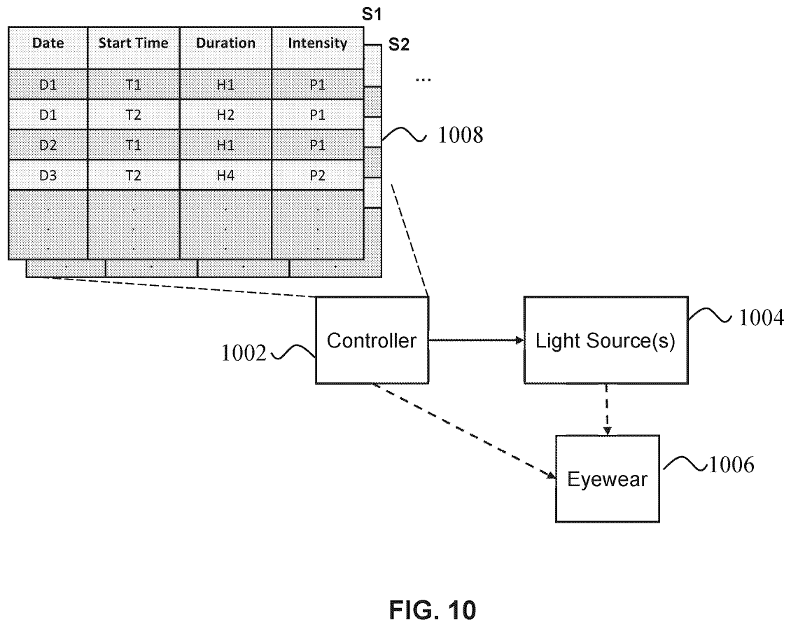

FIG. 10 illustrates an eyewear system for administering GLED in accordance with an example embodiment. The system includes a controller 1002, one or more light sources 1004 that are communicatively coupled to the controller, and an eyewear device. The controller can include, or receive, one or more pre-determined GLED administration schedules 1008 that are used to control the operation of the light source(s) 1004. For example, the pre-determined schedule can include a listing of dates, start times, durations and intensity (or power) levels for administering the light. In some embodiments, a first pre-determined administration schedule, S1, may correspond to light therapy that is to be administered to a first subject, a second pre-determined administration schedule, S2, may correspond to light therapy that is to be administered to a second subject, and so on. In some embodiments, a first pre-determined administration schedule, S1, may correspond to a first condition (e.g., Alzheimer's), a second pre-determined administration schedule, S2, may correspond to a second condition (e.g., PTSD), and so on.

In some embodiment, the one or more light sources 1004 is a light source that provides ambient light for the environment where the subject is located. Examples of the light source 1004 include an LED, a halogen lamp, or a laser light source. In some embodiments, a diffuser (not shown) is included to diffuse the light from the light source 1004 to allow better light distribution. In some embodiments, the light source 1004 is a broadband source (e.g., white light source) that further includes one or more filters that allow only light in the green range of wavelengths (e.g., 500-550 nm) to pass through. In some embodiments, the light source 1004 itself is a green light source that generates light in the green range of wavelengths.

The eyewear 1006 (or generally the eyewear device) can be a pair of glasses, any one of different types of goggles described earlier, or contact lenses. In some embodiments, the eyewear includes a filter to facilitate delivery of light with a particular spectral content to the subject's eyes. For example, the eyewear 1006 can allow green light having a particular wavelength (or range of wavelengths) to pass through to the subject's eyes. In one example implementation, the light source 1004 produces a broadband illumination, and the filter of the eyewear allows light only in the green range of wavelengths (e.g., 500-550 nm) to pass through. As another example, the filter of the eyewear allows light only in a narrow range of green wavelengths (e.g., 520-530 nm) to pass through, when the light source 1004 produces green light in a broader range of wavelengths (e.g., 500-550 nm). The filters may be integrated as part of contact lenses, or lenses and optical components of the eyeglasses or goggles. In some embodiments, the filters are removable, allowing different filters (e.g., suitable for different subjects or different conditions) to be used with the eyewear.

In some embodiments, the controller 1002 is integrated as part of the light source(s) 1004, while in other embodiments, the controller 1002 and the light source(s) 1004 are separate components that can communicate with one another through a wired or wireless connection. In one example embodiment, the controller 1002 is part of a mobile device (e.g., a mobile phone, tablet, etc.) that communicates with the light source(s) 1004 via a cellular, WiFi, or Bluetooth connection. For example, a user (e.g., a physician, a therapist or the subject) may initiate, end or modify green light administration using an application on the mobile device. In some embodiments, the light source(s) 1004 is integrated as part of the eyewear 1006. For example, small LEDs incorporated as part of the eyeglasses or goggles can provide the green light to the subject's eyes. In some embodiments, the controller 1002, may be in communication, or be incorporated at least in-part, with the eyewear 1006. In some embodiments, both the controller 1002 and the light source(s) 1004 are incorporated as part of the eyewear 1006. For instance, the controller 1002 in such a fully integrated implementation may be able to download and store GLED administration schedules through communications with a remote device, and carry out the administration procedure automatically, or upon a receiving a command from the subject (e.g., when the subject wears and turns on the eyewear device, via a voice command, or via activation from a remote connected device).

Non-limiting examples of pre-determined or pre-arranged administration schedules include the following. For cognitive deficits: Fifteen minutes to 8 hours daily using 4-100 lux intensity. Daily exposure is for 3 to 10 weeks, then optional every other night exposure, depending on the severity of the cognitive deficit. Memory impairments: Fifteen minutes to 8 hours daily or nightly using 4-100 lux intensity. Daily exposure is for 3 to 10 weeks, then optional every other night exposure, depending on the severity of the memory impairment. PTSD and anxiety: exposure starts after sunset. Fifteen minutes to 8 hours nightly using 4-100 lux intensity. Nightly exposure for 1 to 8 weeks, then optional every other night exposure, depending on the severity of the PTSD.

One aspect of the disclosed embodiments relates to an eyewear system that includes one or more light sources having emission spectra within a green range of spectra. The eyewear system also includes an eyewear device configured to allow administration of green light to a person wearing the device, and a controller configured to control at least one of: an intensity of green light emission from the one or more light sources, one or more durations of administration of the green light in accordance to a pre-arranged administration schedule. The controller is configured to allow administration of the green light to a person wearing the eyewear device for a first time period and according to the pre-arranged schedule, and administration, for a second time and subsequent periods according to the pre-arranged schedule of administrations.

In one example embodiment, the green light includes a wavelength range of 500 to 550 nanometers. In another example embodiment, the administration comprises administering the green light of 0.001 lux to 500 lux intensity. In yet another example embodiment, the administration allows reducing symptoms associated with posttraumatic stress disorder (PTSD) including reducing anxiety associated with PTSD. In still another example embodiment, the administration for the first time includes administration subsequent to onset of the PTSD symptoms. In one example, embodiment, the administration for the first time, the second time or the subsequent periods spans a period during which a stressful event that triggers the PTSD symptoms is occurring.

According to another example embodiment, the eyewear device is one of eyeglasses or goggles. In yet another example embodiment, the eyewear device includes one or more lenses that only allow wavelengths in the green light spectra to pass therethrough. For example, the one or more lenses are one or more contact lenses. In another example embodiment, the one or more light sources are integrated as part of the eyewear device. In still another example embodiment, the controller is integrated as part of the eyewear device. In another example embodiment, the one or more light sources are configured to administer the green light by illuminating an environment around the person.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

It is understood that the various disclosed embodiments may be implemented individually, or collectively, using devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes

Only a few implementations and examples are described and other implementations, enhancements and variations can be made based on what is described and illustrated in this patent document.

1. A method for managing or minimizing cognitive deficits or memory impairments, comprising:
administering, for a first time period and according to a pre-arranged schedule of administrations, light with one or more wavelengths in the range between approximately 515 nm and approximately 535 nm to a retina of a subject; and
administrating, for a second time and subsequent periods according to the pre-arranged schedule of administrations, light with the one or more wavelengths in the range between approximately 515 nm and approximately 535 nm to the retina of the subject,
wherein the first, the second and the subsequent time periods occur on separate days, and wherein the pre-arranged schedule of administrations includes administrations that span over at least one week.

2. The method of claim 1, wherein the first, the second and the subsequent time periods each occur on consecutive days.

3. The method of claim 1, wherein the first, the second and the subsequent time periods occur on consecutive days for more than one week.

4. (canceled)

5. The method of claim 1, wherein the administering comprises administering light of 0.001 lux to 1000 lux.

6. (canceled)

7. The method of claim 1, wherein said administering includes contacting an eye of the subject with a material that allows light between approximately 515 nm and approximately 535 nm to enter the retina of the subject.

8. (canceled)

9. The method of claim 1, wherein the subject is affected by a neurogenerative disease, and wherein administering the light for the first, the second and the subsequent time periods that span over at least one week is operable to mitigate cognitive deficits or memory impairments associated with the neurogenerative disease via modulating activation of microglia.

10. (canceled)

11. A method for modulating microglia activation in a subject, comprising:
administering, for a first time period and according to a pre-arranged schedule of administrations, light with one or more wavelengths in a green range of wavelengths to a retina of the subject; and
administrating, for a second time and subsequent periods according to the pre-arranged schedule of administrations, light with the one or more wavelengths in the green range of wavelengths to the retina of the subject,
wherein the first, the second and the subsequent time periods occur on separate days, and wherein the pre-arranged schedule of administrations includes administrations that span over at least one week.

12. The method of claim 11, wherein modulating the microglia activation is operable to mitigate an inflammatory response associated with severe acute respiratory syndrome coronavirus-2 (SARS-CoV-2).

13. The method of claim 11, wherein modulating the microglia activation is operable to mitigate symptoms associated with one or more of: depression, anxiety, or postpartum depression.

14. A device for microglia activation, comprising:
one or more light sources having emission spectra within a range from approximately 515 nm to approximately 535 nm associated with green light; and
a controller configured to control at least one of: intensity of light emission or duration of light emission of the one or more light sources,
wherein the device is configured to allow administration of the green light to a person wearing the device.

15. The device of claim 14, wherein the device includes one of: goggles, ski goggles, virtual reality goggles, augmented reality goggles, a pair of eyeglasses, or a contact lens.

16. A method for reducing symptoms associated with posttraumatic stress disorder (PTSD), comprising:
administering to a subject, for a first time period and according to a pre-arranged schedule of administrations, green light including a wavelength of 525 nanometer; and
administrating to the subject, for a second time and subsequent periods according to the pre-arranged schedule of administrations, the green light including the wavelength of 525 nanometer,
wherein the first, the second and the subsequent time periods occur on separate days, and wherein the pre-arranged schedule of administrations includes administrations that span from 15 minutes to 4 hours daily or every other day.

17. The method of claim 16, wherein the green light includes a range of 500 to 550 nanometers.

18. The method of claim 16, wherein the administering comprises administering the green light of 0.001 lux to 500 lux intensity.

19. The method of claim 16, wherein the first time, the second time and for the subsequent periods according to the pre-arranged schedule includes administering the green light including the wavelength of 525 nanometer for fifteen minutes to 8 hours nightly using an intensity in the range 4 to 100 lux, for a period of one to 8 weeks.

20. The method of claim 16, wherein reducing symptoms associated with posttraumatic stress disorder (PTSD) includes reducing anxiety associated with PTSD, wherein the administering for the first time includes administering subsequent to onset of the PTSD symptoms.

21. (canceled)

22. (canceled)

23. (canceled)

24. (canceled)

25. (canceled)

26. The method of claim 16, comprising delivering the green light through a light source that illuminates an environment around the subject.

27. An eyewear system, comprising:
one or more light sources having emission spectra within a green range of spectra;
an eyewear device configured to allow administration of green light to a person wearing the device; and
a controller configured to control at least one of: an intensity of green light emission from the one or more light sources, one or more durations of administration of the green light in accordance to a pre-arranged administration schedule, wherein
the controller is configured to allow administration of the green light to the person wearing the eyewear device for a first time period and according to the pre-arranged schedule, and administration, for a second time and subsequent periods according to the pre-arranged schedule of administrations.

28. The system of claim 27, wherein the green light includes a wavelength range of 500 to 550 nanometers.

29. (canceled)

30. (canceled)

31. (canceled)

32. (canceled)

33. The system of claim 27, wherein the eyewear device is one of eyeglasses or goggles.

34. The system of claim 27, wherein the eyewear device includes one or more lenses that only allow wavelengths in the green light spectra to pass therethrough.

35. The system of claim 34, wherein the one or more lenses are one or more contact lenses.

36. The system of claim 27, wherein the one or more light sources are integrated as part of the eyewear device.

37. (canceled)

38. The system of claim 27, wherein the one or more light sources are configured to administer the green light by illuminating an environment around the person.
